(19) United States
(12) Patent Application Publication
(10) Pub. No.: US 2023/0197257 A1
(43) Pub. Date: Jun. 22, 2023

(54) INFORMATION PROCESSING APPARATUS, INFORMATION PROCESSING METHOD, AND NON-TRANSITORY COMPUTER-READABLE STORAGE MEDIUM

(71) Applicant: NEC Corporation, Minato-ku, Tokyo (JP)

(72) Inventor: Ryoji NOTOMI, Tokyo (JP)

(73) Assignee: NEC Corporation, Minato-ku, Tokyo (JP)

(21) Appl. No.: 17/925,886

(22) PCT Filed: May 25, 2020
(86) PCT No.: PCT/JP2020/020482

§ 371 Date: Nov. 17, 2022

(51) Int. Cl. G16H 40/20; G16H 40/67; G06F 21/32

(52) U.S. Cl. CPC G16H 40/20; G16H 40/67; G06F 21/32

(57) ABSTRACT

An information processing apparatus (100) includes an acquisition unit (102) acquiring biometric information of a visiting caregiver, a decision unit (104) reading a qualification of the caregiver corresponding to the acquired biometric information from a qualified person database (50) storing biometric information and a qualification in association with each other and deciding whether the qualification satisfies a criterion, and a storage processing unit (106) causing a management information database (110) to store the decision result.

### TECHNICAL FIELD

The present invention relates to a home-visit caregiving service system, an information processing apparatus, an information processing method, and a program.

### BACKGROUND ART

More weight has been placed on home care due to a declining birthrate and aging population, saturated hospitalization facilities and nursing care facilities, and the like; and needs for home-visit caregiving services and remote healthcare are surmised to increase accordingly.

Patent Document 1 describes a care assistance system outputting a future care plan by using a care record of a care recipient. Further, the system allows a family of the care recipient to browse the care record from a family terminal.

Patent Document 2 describes a system classifying care target persons into a plurality of care types, subsequently selecting a recommended care plan, based on the classified care type, providing a main menu based on the care type, and allowing selection of a desired care-related service and a desired product from the menu.

On the other hand, social problems such as a theft, violence, and impersonation by an unqualified person have occurred in home-visit caregiving. Patent Document 3 describes a technology for checking the identity of a visitor and preventing a criminal act by acquiring biometric information of the visitor near an entrance of a building and performing personal authentication of a caregiver by checking the biometric information against biometric information of a caregiver planned to make a visit.

Patent Document 4 describes acquiring a video image of an action of a caregiver with a surveillance camera, comparing the image with video image data of a sample care action, and when the difference is large, recording the image as there is a high possibility of a wrongdoing and making notification to a person in charge. When receiving the notification, the person in charge can check existence of a wrongdoing by checking the record.

### RELATED DOCUMENTS

### Patent Documents

Patent Document 1: Japanese Patent Application Publication No. 2018-92493

Patent Document 2: Japanese Patent Application Publication No. 11-149499

Patent Document 3: Japanese Patent Application Publication No. 2003-114923

Patent Document 4: Japanese Patent Application Publication No. 2007-4405

### SUMMARY OF INVENTION

### Technical Problem

In general, a home-visit caregiver starts care work after giving a verbal description and a greeting to a care recipient in home-visit caregiving. On the other hand, while a qualification is required for becoming a home-visit caregiver, checking existence of the qualification is practically difficult in home-visit caregiving.

The present invention has been made in view of the aforementioned circumstances, and an object thereof is to enable checking of whether a home-visit caregiver is qualified in home-visit caregiving.

### Solution to Problem

In order to solve the aforementioned issues, the following configurations are employed in aspects of the present invention, respectively.

A first aspect relates to an information processing apparatus.

An information processing apparatus according to the first aspect includes:

an acquisition unit that acquires biometric information of a visiting caregiver;

a decision unit that reads a qualification of the caregiver corresponding to the acquired biometric information from a storage unit that stores the biometric information and a qualification in association with each other and decides whether the qualification satisfies a criterion; and

a storage processing unit that causes a storage unit to store the decision result.

A second aspect relates to an information processing method executed by at least one computer.

An information processing method according to the second aspect includes, by an information processing apparatus:

acquiring biometric information of a visiting caregiver;

reading a qualification of the caregiver corresponding to the acquired biometric information from a storage unit that stores the biometric information and a qualification in association with each other and deciding whether the qualification satisfies a criterion; and

causing a storage unit to store the decision result.

A third aspect relates to a home-visit caregiving service system.

A home-visit caregiving service system according to the third aspect includes:

a biometric information acquisition apparatus that acquires biometric information of a caregiver at a place being visited; and

a server apparatus, wherein

the server apparatus includes:
an acquisition unit that acquires biometric information of the visiting caregiver by using the biometric information acquisition apparatus;
a decision unit that reads a qualification of the caregiver corresponding to the acquired biometric information from a storage unit that stores the biometric information and a qualification in association with each other and decides whether the qualification satisfies a criterion; and
a storage processing unit that causes a storage unit to store the decision result.

Note that another aspect of the present invention may be a program causing at least one computer to execute the method according to the aforementioned second aspect or may be a computer-readable storage medium on which such a program is recorded. The storage medium includes a non-transitory tangible medium.

The computer program includes a computer program code causing a computer to implement the information processing method on the information processing apparatus when being executed by the computer.

Note that any combination of the components described above, and representations of the present invention converted among a method, an apparatus, a system, a storage medium, a computer program, and the like are also valid as embodiments of the present invention.

Further, various components of the present invention do not necessarily need to be individually independent, and, for example, a plurality of components may be formed as a single member, a plurality of members may form a single component, a certain component may be part of another component, and part of a certain component may overlap with part of another component.

Further, while a plurality of procedures are described in a sequential order in the method and the computer program according to the present invention, the order of description does not limit the order in which the plurality of procedures are executed. Therefore, when the method and the computer program according to the present invention are implemented, the order of the plurality of procedures may be changed without affecting the contents.

Furthermore, a plurality of procedures in the method and the computer program according to the present invention are not limited to be executed at timings different from each other. Therefore, for example, a certain procedure may be executed during execution of another procedure, and an execution timing of a certain procedure and an execution timing of another procedure may overlap with each other in part or in whole.

### Advantageous Effects of Invention

Each of the aforementioned aspects enables checking of whether a home-visit caregiver is qualified in home-visit caregiving.

### BRIEF DESCRIPTION OF THE DRAWINGS

FIG. 1 is a functional block diagram logically illustrating a configuration of an information processing apparatus according to an example embodiment of the present invention.

FIG. 2 is a diagram illustrating an example of a data structure of a qualified person database.

FIG. 3 is a diagram illustrating an example of a data structure of a management information database.

FIG. 4 is a block diagram illustrating a hardware configuration of a computer providing the information processing apparatus in FIG. 1.

FIG. 5 is a flowchart illustrating an operation example of the information processing apparatus according to the present example embodiment.

FIG. 6 is a diagram illustrating a configuration example of a home-visit caregiving service system according to an example embodiment of the present invention.

FIG. 7 is a diagram illustrating examples of a data structure of qualification information.

FIG. 8 is a flowchart illustrating an operation example of the home-visit caregiving service system according to the present example embodiment.

FIG. 9 is a diagram illustrating examples of a data structure of care plan information.

FIG. 10 is a flowchart illustrating an example of details of decision processing.

FIG. 11 is a flowchart illustrating another example of details of the decision processing.

FIG. 12 is a functional block diagram logically illustrating a configuration of a server apparatus according to the present example embodiment.

FIG. 13 is a functional block diagram logically illustrating a configuration of a server apparatus according to the present example embodiment.

FIG. 14 is a functional block diagram logically illustrating a configuration of a server apparatus according to the present example embodiment.

FIG. 15 is a diagram illustrating an example of a data structure of care history information.

FIG. 16 is a functional block diagram logically illustrating a configuration of a server apparatus according to the present example embodiment.

### EXAMPLE EMBODIMENT

Example embodiments of the present invention will be described below by using drawings. Note that, in every drawing, similar components are given similar signs, and description thereof is not included as appropriate. Further, in every diagram, a configuration of a part unrelated to the essence of the present invention is not included and is not illustrated.

In the example embodiments, “acquisition” includes at least either of an apparatus getting data or information stored in another apparatus or a storage medium (active acquisition), and an apparatus inputting data or information output from another apparatus to the apparatus (passive acquisition). Examples of the active acquisition include making a request or an inquiry to another apparatus and receiving a response, and readout by accessing another apparatus or a storage medium. Further, examples of the passive acquisition include reception of distributed (or, for example, transmitted or push notified) information. Furthermore, “acquisition” may refer to acquisition by selection from received data or information, or selective reception of distributed data or information.

### First Example Embodiment

### <Functional Configuration Example>

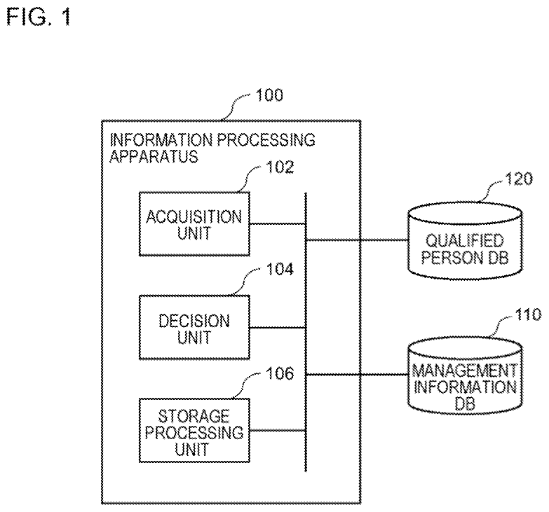

FIG. 1 is a functional block diagram logically illustrating a configuration of an information processing apparatus 100 according to an example embodiment of the present invention. The information processing apparatus 100 includes an acquisition unit 102, a decision unit 104, and a storage processing unit 106. The information processing apparatus 100 is connected to a management information database 110 and a qualified person database 120. The management information database 110 and the qualified person database 120 may be storage apparatuses included inside the information processing apparatus 100, may be apparatuses separate from the information processing apparatus 100, or may be a combination of the two. Further, the management information database 110 and the qualified person database 120 are not limited to a database structure.

Thee acquisition unit 102 acquires biometric information of a visiting caregiver.

The decision unit 104 reads a qualification of a caregiver corresponding to the acquired biometric information from the qualified person database 120 storing biometric information and a qualification in association with each other and decides whether the qualification satisfies a criterion.

The storage processing unit 106 causes the management information database 110 to store the decision result.

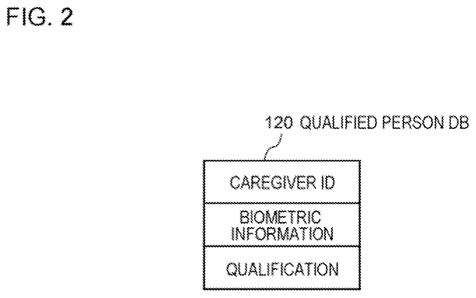

FIG. 2 is a diagram illustrating an example of a data structure of the qualified person database 120. The qualified person database 120 stores identification information of a caregiver CG (hereinafter also referred to as a caregiver ID), biometric information of the caregiver CG, and qualification information in association with one another.

Qualification information of a caregiver CG is information indicating a level of a care-related qualification held by the caregiver CG.

A level of a qualification is indicated by the type or the grade of the qualification. For example, information indicating a level of a qualification is information indicating at least one qualification out of a care worker, a working-level care worker training, a beginning care staff training, a seriously-ill patient home helper, an intense behavior disorder supporter, a life support worker training, a sputum suction training, an activity support worker, a mobile care worker, a recreational caregiver first grade or second grade, a nurse, and a senior nutritionist but is not limited thereto.

The qualified person database 120 holds information about a qualified person for each of various qualifications. As for information about a qualified person, for example, the information can be acquired from an issuer of a qualification and be registered in the qualified person database 120. Therefore, reliability of qualification information is higher compared with information declared by a caregiver CG. Further, latest information is always registered in the qualified person database 120. However, qualification information of a caregiver CG may be registered in the qualified person database 120, based on a declaration from the caregiver CG or a care provider.

When a qualification of a caregiver CG satisfies a required level as a result of decision by the decision unit 104, information indicating that an eligible person is visiting, such as an eligible person flag, is set to 1. When the qualification of the caregiver CG does not satisfy the required level, information indicating that an ineligible person is visiting, such as the eligible person flag, is reset to 0.

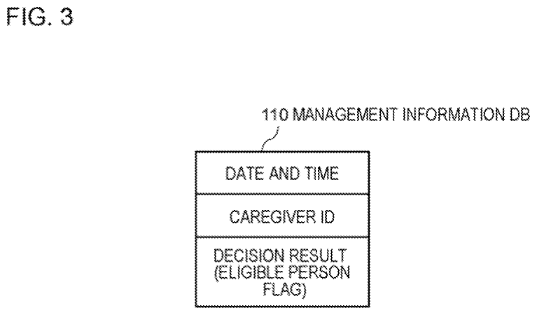

FIG. 3 is a diagram illustrating an example of a data structure of the management information database 110. The management information database 110 stores a date and time of a visit to a care recipient CP by a caregiver CG, identification information of the caregiver CG (a caregiver ID), and a decision result by the decision unit 104 in association with one another.

### <Hardware Configuration Example>

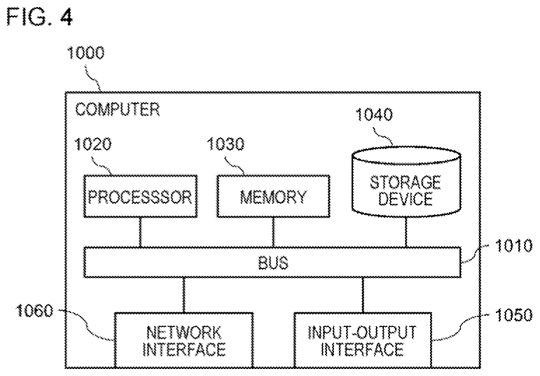

FIG. 4 is a block diagram illustrating a hardware configuration of a computer 1000 providing the information processing apparatus 100 in FIG. 1.

The computer 1000 includes a bus 1010, a processor 1020, a memory 1030, a storage device 1040, an input-output interface 1050, and a network interface 1060.

The bus 1010 is a data transmission channel for the processor 1020, the memory 1030, the storage device 1040, the input-output interface 1050, and the network interface 1060 to transmit and receive data to and from one another. Note that the method for interconnecting the processor 1020 and other components is not limited to a bus connection.

The processor 1020 is a processor provided by a central processing unit (CPU), a graphics processing unit (GPU), or the like.

The memory 1030 is a main storage provided by a random access memory (RAM) or the like.

The storage device 1040 is an auxiliary storage provided by a hard disk drive (HDD), a solid state drive (SSD), a memory card, a read only memory (ROM), or the like. The storage device 1040 stores program modules providing the functions of the information processing apparatus 100 (such as the acquisition unit 102, the decision unit 104, the storage processing unit 106, and a providing unit 108, a notification unit 112, and an acceptance unit 114 to be described later). By reading each program module into the memory 1030 and executing the program module by the processor 1020, each function related to the program module is provided. The storage device 1040 may store data in the information processing apparatus 100, such as care information by qualification 130 and care plan information 140 to be described later. Further, the storage device 1040 may also store data in the management information database 110 and the qualified person database 120.

A program module may be recorded on a storage medium. The storage medium on which the program module is recorded includes a non-transitory tangible media usable by the computer 1000; and a program code readable by the computer 1000 (processor 1020) may be embedded in the medium.

The input-output interface 1050 is an interface for connecting the computer 1000 to various types of input-output equipment. The input-output interface 1050 also functions as a communication interface performing short-distance wireless communication such as Bluetooth (registered trademark) and/or near field communication (NFC).

The network interface 1060 is an interface for connecting the computer 1000 to a communication network. Examples of the communication network include a local area network (LAN) and a wide area network (WAN). The method for connecting the network interface 1060 to the communication network may be a wireless connection or a wired connection.

Then, the computer 1000 is connected to required equipment (such as output apparatuses such as a display and a printer, and an authentication terminal 5 and a data coordination apparatus 200 to be described later) through the input-output interface 1050 or the network interface 1060.

Each component in the information processing apparatus 100 in FIG. 1 is provided by any combination of hardware and software of the computer 1000 in FIG. 4. Then, it should be understood by a person skilled in the art that various modifications to the providing method and the apparatus can be made. A functional block diagram illustrating a server apparatus 100 according to each example embodiment represents logical function-based blocks rather than a hardware-based configuration.

Further, each of the authentication terminal 5 and the coordination apparatus 200 to be described later is provided by any combination of hardware and software of the computer 1000 in FIG. 4.

### <Operation Example>

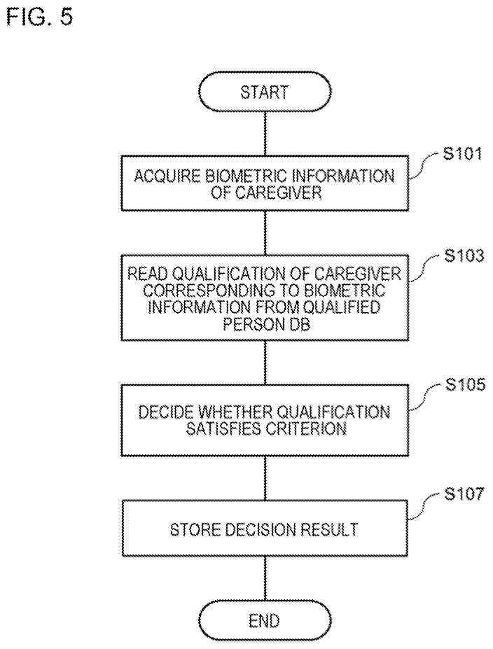

FIG. 5 is a flowchart illustrating an operation example of the information processing apparatus 100 according to the present example embodiment.

First, the acquisition unit 102 acquires biometric information of a visiting caregiver (Step S101). Then, the decision unit 104 reads a qualification of the caregiver corresponding to the acquired biometric information from the qualified person database 120 (FIG. 2) (Step S103). Then, the decision unit 104 decides whether the read qualification satisfies a criterion (Step S105). Then, the storage processing unit 106 causes the management information database 110 (FIG. 3) to store the decision result (Step S107).

According to the present example embodiment, the acquisition unit 102 acquires biometric information of a caregiver CG, the decision unit 104 reads a qualification of a caregiver CG corresponding to the biometric information from the qualified person database 120 and decides whether the qualification satisfies a criterion, and the storage processing unit 106 stores the decision result into the management information database 110. With the configuration, a qualification of a caregiver CG visiting a house of a care recipient CP can be acquired from biometric information of the caregiver CG, and a decision result of whether a qualified person has visited can be left as a record. Accordingly, whether a home-visit caregiver is qualified can be checked in home-visit caregiving, and a wrongful act such as impersonating a caregiver CG can be prevented. Alternatively, a wrongful act can be found. Furthermore, a record in the management information database 110 may serve as an evidence of a wrongful act.

### Second Example Embodiment

### <System Overview>

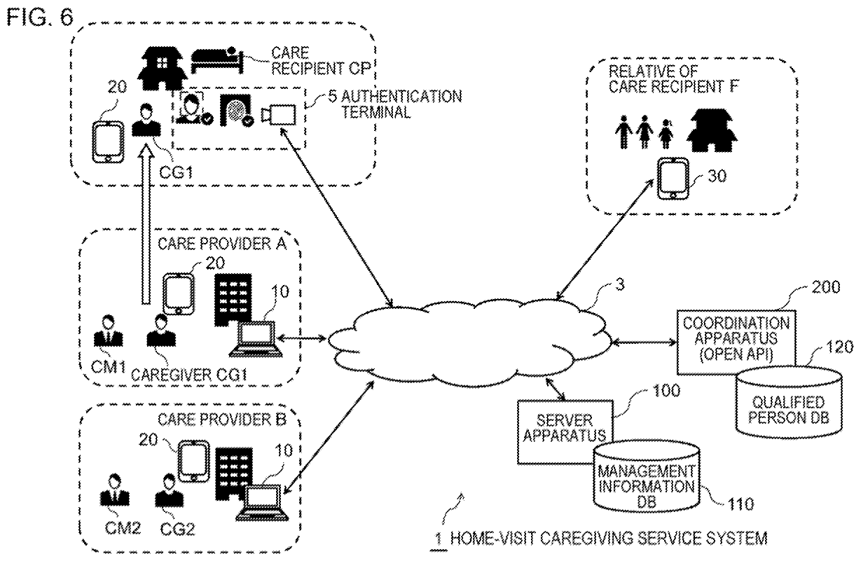

FIG. 6 is a diagram illustrating a configuration example of a home-visit caregiving service system 1 according to an example embodiment of the present invention. In the home-visit caregiving service, a caregiver CG is dispatched to a home of a care recipient CP, based on a care plan drawn up by a care manager CM at each care provider. The home-visit caregiving service system 1 manages a home-visit caregiving service provided for the care recipient CP by the caregiver CG.

A caregiver CG is a so-called home helper and is a home-visit caregiver visiting a house of a care recipient CP and performing work related to care of the care recipient CP in accordance with a care plan. For example, it is assumed in the present example embodiment that a care recipient CP lives in a house separate from that of a relative F or that a person living together with the care recipient CP is absent. Furthermore, not only a case of a care recipient CP being at home by himself/herself when a caregiver CG makes a visit but also other cases such as a case of a person living together excluding a care recipient CP being an aged person, a handicapped person, a minor, or the like are assumed. Specifically, a case that when a caregiver CG makes a visit, a care recipient CP himself/herself, or a person living together, a relative F, or the like is in a condition of not being able to notice a visit by a person wrongfully impersonating the caregiver CG is assumed.

The home-visit caregiving service system 1 includes a server apparatus 100 corresponding to the information processing apparatus 100 according to the first example embodiment, an authentication terminal 5, and a coordination apparatus 200. The server apparatus 100 is connected to a management information database 110. The coordination apparatus 200 is connected to a qualified person database 120. The authentication terminal 5 is installed at a house of a care recipient CP and is connected to the server apparatus 100 through a communication network 3.

Furthermore, the home-visit caregiving service system 1 may include a care provider terminal 10 for each care provider, a caregiver terminal 20 for a caregiver CG, and a care recipient terminal 30 for a relative F of a care recipient CP.

Examples of the care provider terminal 10 and the care recipient terminal 30 include a personal computer, a tablet terminal, and a smartphone. The caregiver terminal 20 is preferably portable and is, for example, a tablet terminal or a smartphone. Each of the care provider terminal 10, the caregiver terminal 20, and the care recipient terminal 30 is also provided by the computer 1000 in FIG. 4.

For example, the coordination apparatus 200 is provided by an open application programming interface (API). Information about a qualified person is registered for each of various caregiver-related qualifications in the qualified person database 120, and biometric information of each caregiver is also registered. In response to an inquiry from the server apparatus 100, the coordination apparatus 200 returns inquired data from data registered in the qualified person database 120.

Specifically, the coordination apparatus 200 accepts an inquiry about qualification information of a caregiver CG from the server apparatus 100 with biometric information of the caregiver CG as a key, determines the caregiver CG by checking the biometric information against biometric information registered in the qualified person database 120, and returns qualification information held by the determined caregiver CG to the server apparatus 100.

### <Functional Configuration Example>

The server apparatus (information processing apparatus) 100 according to the present example embodiment includes an acquisition unit 102, a decision unit 104, and a storage processing unit 106 that are the same as those in the information processing apparatus 100 according to the first example embodiment in FIG. 1. Note that the server apparatus 100 according to the present example embodiment may be combined with at least one of the configurations according to other example embodiments without contradicting each other.

The authentication terminal 5 is an apparatus for acquiring biometric information of a caregiver CG visiting a house of a care recipient CP and performing authentication processing.

For example, biometric information includes a feature value of at least one of a face, an iris, a vein, a pinna, a fingerprint, a voiceprint, a gait, and a body-build (such as a height, a shoulder width, a stature, or a skeletal structure). The authentication terminal 5 extracts biometric information (such as a facial image) or a feature value of the biometric information from an image generated by a camera. Alternatively, the authentication terminal 5 may acquire a voiceprint from a voice collected from a microphone (unillustrated).

The authentication terminal 5 may further acquire biometric information of the caregiver CG and perform the authentication processing when the caregiver CG ends the care. The authentication terminal 5 may transmit times when biometric information is acquired to the server apparatus 100 as a starting time and an ending time of the care. The storage processing unit 106 in the server apparatus 100 stores the starting time and the ending time that are received from the authentication terminal 5 into the management information database 110. Furthermore, the storage processing unit 106 may compute work hours of the caregiver CG for the day from the starting time and the ending time and store the work hours into the management information database 110.

The acquisition unit 102 receives, through the communication network 3, biometric information of a caregiver CG transmitted from the authentication terminal 5.

The decision unit 104 inquires of the coordination apparatus 200 about a qualification of a caregiver CG with a feature value of biometric information received from the authentication terminal 5 as a key. The decision unit 104 receives the qualification of the caregiver CG from the coordination apparatus 200. The decision unit 104 decides whether the qualification satisfies a criterion.

Some type of care may require a predetermined qualification depending on the content of the care. Therefore, a performable care item is associated with each level of a qualification. Alternatively, a level of a qualification is set for each care item.

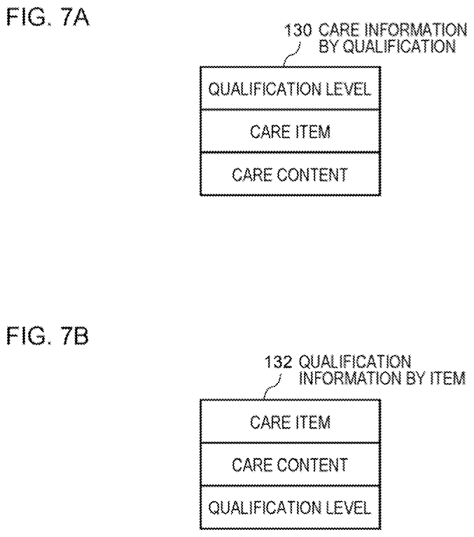

FIG. 7 is a diagram illustrating examples of a data structure of qualification information. FIG. 7A is an example of care information by qualification 130, and FIG. 7B is an example of qualification information by item 132.

A performable care item is associated with each level of a qualification (denoted by “QUALIFICATION LEVEL” in the diagram) in the care information by qualification 130 in FIG. 7A. While a care content may be further associated, a care content is not necessarily required. For each care item, a level of a qualification required for performing the care is associated in the qualification information by item 132 in FIG. 7B. While a care content may be further associated, a care content is not necessarily required.

Further, the care information by qualification 130 may be stored in the qualified person database 120 instead of the management information database 110. When the decision unit 104 inquires of the coordination apparatus 200 about qualification information of a caregiver CG, the coordination apparatus 200 may return a level of a qualification along with a care item performable with the qualification to the server apparatus 100.

Examples of a care content includes physical care, life support, mental care, and consultation, guidance, and advice to a family of a care recipient CP.

As for a care item in each care content, examples of physical care include assistance for changing of clothes, assistance for eating, assistance for bathing, assistance for excretion, assistance for rising from a bed, and assistance for walking. Examples of life support include cooking, preparation of a meal, cleaning a room, keeping things in order, washing, and shopping. Examples of mental care include being a companion, holding a recreational event, prompting mingling with neighbors and other service users, and consultation, guidance, and advice for care. Alternatively, measures performable by a nurse, such as injection, intravenous dripping, and collection of blood may also be included.

Before a caregiver CG visits a care recipient CP, daily care items are preplanned by a care manager CM, and a care plan is generated. In general, after determining a level of a qualification of a caregiver CG corresponded to the care items planned in the care plan, a caregiver CG visiting on an appointed day is selected, and a schedule is drawn up. However, a caregiver CG other than the caregiver CG planned to visit may unexpectedly make a visit, or the care content may be changed. Furthermore, a wrongdoing of another person impersonating the caregiver CG planned to visit making a visit may occur.

According to the present example embodiment, a level of a qualification of a caregiver CG is set based on a care plan of the appointed day. The decision unit 104 decides whether a qualification of a caregiver CG satisfies the level of the qualification of the care plan of the appointed day. A decision method by the decision unit 104 will be described later.

### <Operation Example>

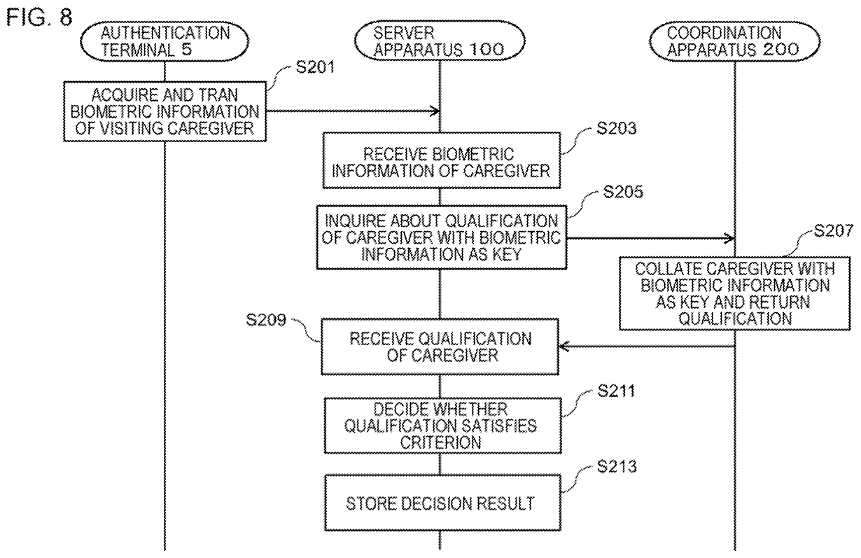

FIG. 8 is a flowchart illustrating an operation example of the home-visit caregiving service system 1 according to the present example embodiment.

When a caregiver CG visits a house of a care recipient CP, the authentication terminal 5 provided at the entrance of the house of the care recipient CP acquires biometric information of the caregiver CG (Step S201). The acquired biometric information is transmitted to the server apparatus 100 from the authentication terminal 5, and the acquisition unit 102 in the server apparatus 100 receives the biometric information of the caregiver CG (Step S203).

For example, a facial image is generated by capturing an image of the face of the caregiver CG with a camera in the authentication terminal 5, and the facial image is transmitted to the server apparatus 100. The acquisition unit 102 in the server apparatus 100 receives the facial image from the authentication terminal 5. The decision unit 104 inquires of the coordination apparatus 200 about qualification information of the caregiver CG with the received facial image or a feature value extracted from the facial image as a key (Step S205).

The coordination apparatus 200 collates the biometric information of the caregiver CG with biometric information in the qualified person database 120 in FIG. 2. The coordination apparatus 200 reads qualification information of a caregiver CG associated with biometric information in the qualified person database 120 matching the biometric information of the caregiver CG by a threshold value or greater from the qualified person database 120 and returns the qualification information to the server apparatus 100 (Step S207).

Then, the decision unit 104 in the server apparatus 100 receives the qualification of the caregiver CG from the coordination apparatus 200 (Step S209). Then, the decision unit 104 decides whether the received qualification satisfies a criterion (Step S211).

The care plan information 140 is used in the decision processing in Step S211. At least two data structures as follows can be considered as a data structure of the care plan information 140, and a procedure for the decision processing varies with the data structure. Details will be described below.

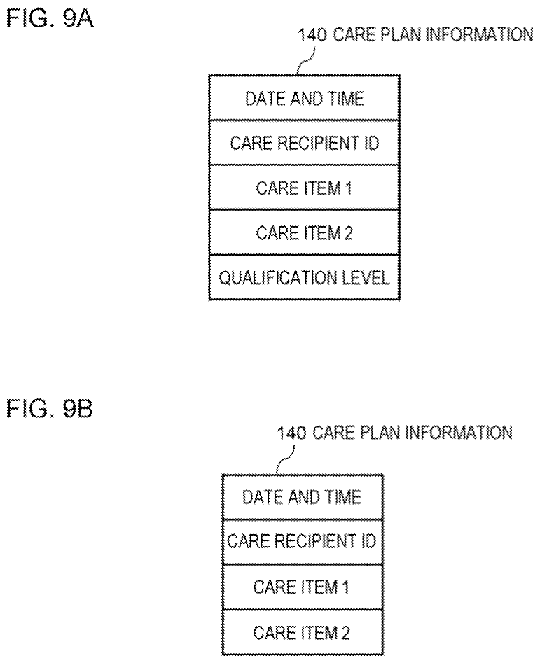

FIG. 9 is a diagram illustrating examples of a data structure of the care plan information 140. A date and time when care is performed, identification information of a care recipient CP (hereinafter also referred to as a care recipient ID), at least one planned care item, and a level of a qualification, the level being required for performing care represented by the planned care item, are associated with one another in the care plan information 140 in FIG. 9A. While two care items are included in the example in FIG. 9A, the number of care items is not limited to two.

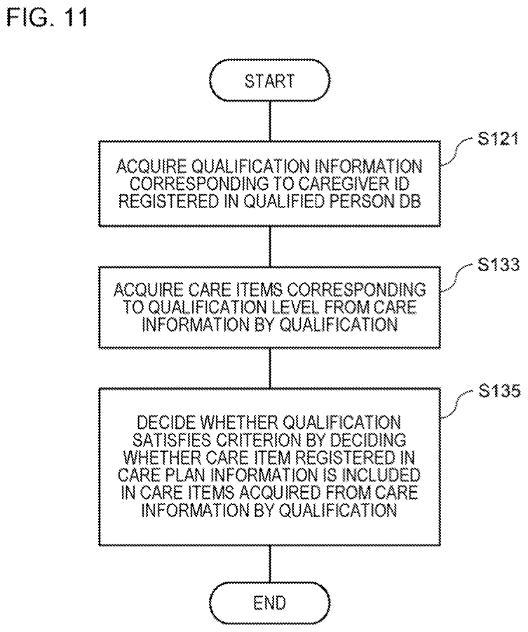

A level of a qualification, the level being required for performing care represented by a planned care item, is not associated in an example of the care plan information 140 in FIG. 9B. The decision unit 104 refers to another piece of information and decides whether a qualification of the caregiver CG satisfies a criterion in this example. Details of the decision processing procedure performed here will be described later. At least the following two procedures (FIG. and FIG. 11) can be considered as the details.

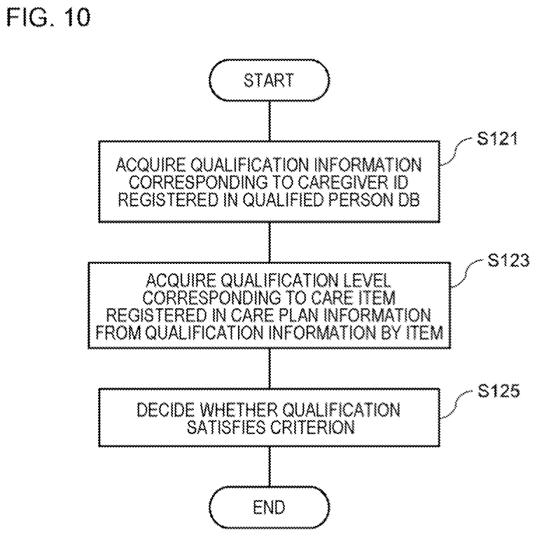

In one example, as illustrated in FIG. 10, the decision unit 104 first reads and acquires qualification information corresponding to identification information of a caregiver CG registered in the care plan information 140 (Step S121). Furthermore, the decision unit 104 reads and acquires a qualification level corresponding to a care item registered in the care plan information 140 in FIG. 9B from the qualification information by item 132 in FIG. 7B (Step S123). Then, the decision unit 104 decides whether a level of a qualification, the level being indicated by the qualification information of the caregiver CG acquired in Step S121, satisfies a criterion for the qualification level acquired in Step S123 (Step S125).

In another example, as illustrated in FIG. 11, the decision unit 104 first acquires qualification information of a caregiver CG, similarly to FIG. 10 (Step S121). Then, the decision unit 104 reads and acquires care items corresponding to a level of a qualification, the level being indicated by the qualification information of the caregiver CG acquired in Step S121, from the care information by qualification 130 in FIG. 7A (Step S133). Then, the decision unit 104 decides whether a care item registered in the care plan information 140 in FIG. 9B is included in the care items acquired in Step S133 (Step S135).

When the care item registered in the care plan information 140 in FIG. 9B is included in the care items acquired in Step S133, the qualification of the caregiver CG is decided to satisfy a level of the care plan of the appointed day. On the other hand, when the care item registered in the care plan information 140 in FIG. 9B is not included in the care items acquired in Step S133, the qualification of the caregiver CG is decided not to satisfy the level required for the care plan.

After such decision, the storage processing unit 106 stores the decision result in Step S211 into the management information database 110 (Step S213).

The present example embodiment provides effects similar to those provided by the aforementioned example embodiment and further enables acquisition of reliable information since the decision unit 104 can acquire qualification information of a caregiver CG by using the coordination apparatus 200 (open API).

### Third Example Embodiment

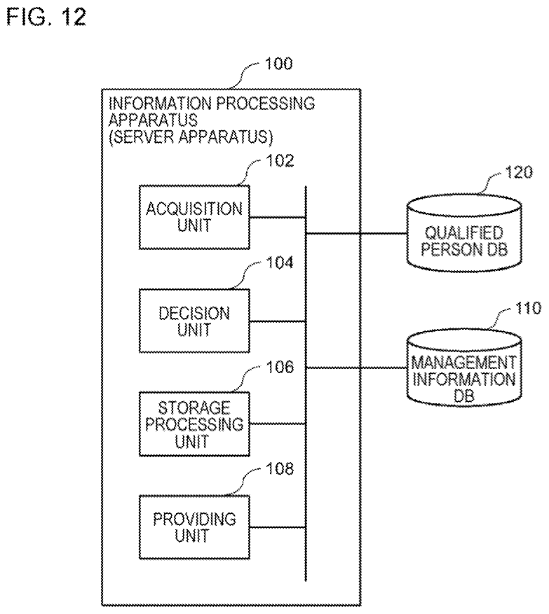

FIG. 12 is a functional block diagram logically illustrating a configuration of a server apparatus (information processing apparatus) 100 according to the present example embodiment. The server apparatus 100 according to the present example embodiment is similar to the aforementioned example embodiment except for being configured to determine and provide an addable caregiving service corresponding to a qualification of a caregiver CG.

### <Functional Configuration Example>

The server apparatus 100 includes an acquisition unit 102, a decision unit 104, and a storage processing unit 106 that are the same as those in the information processing apparatus 100 according to the first example embodiment in FIG. 1 and further includes a providing unit 108. The example in FIG. 12 illustrates an example combined with the configuration of the information processing apparatus 100 according to the first example embodiment in FIG. 1. Note that the server apparatus 100 according to the present example embodiment may be combined with at least one of the configurations according to other example embodiments without contradicting each other.

The providing unit 108 determines and provides an addable service corresponding to the qualification of the caregiver CG by using a level of the qualification, the level being set for each care content, and a care plan.

The providing unit 108 determines an addable service corresponding to the qualification of the caregiver CG by using the care information by qualification 130 in FIG. 7. For example, when a plurality of services are determined, a list may be output. Specifically, the providing unit 108 may extract a service permitted by the qualification of the caregiver CG and determine the difference between a service planned for the today and the extracted service as an addable service. Alternatively, after acceptance of selection of a service which, based on the condition of a care recipient CP on the today, a caregiver CG considers to be preferably performed (although the service is not included in a plan of the today), whether the service is permitted by a qualification of the caregiver CG may be decided, and a permitted service may be determined.

For example, the providing unit 108 displays information about an addable service on a display on a caregiver terminal 20 of a caregiver CG or a care recipient terminal 30 of a relative F. The providing unit 108 may cause a printer connected to a care provider terminal 10 of a care provider to print out information about an addable service. The caregiver CG may provide information displayed on the caregiver terminal 20 for the care recipient CP himself/herself or the relative F and recommend an additional service.

The providing unit 108 can determine an addable service on the appointed day of a visit by a caregiver CG and provide the service for a care recipient CP or a relative F of the care recipient CP on the spot.

In each of the caregiver terminal 20 and the care recipient terminal 30, user preregistration is performed in order to use a home-visit caregiving service system 1, and authentication information such as user identification information (hereinafter also referred to as a user ID) and a password for logging into the home-visit caregiving service system 1 are registered in advance.

For example, in order to use a service provided by the home-visit caregiving service system 1, a method of using an application and a method of using a browser are considered. For example, a service can be used by installing and starting an application for using the home-visit caregiving service system 1 on the caregiver terminal 20 and the care recipient terminal 30 and logging in by inputting a user ID and a password from a menu screen. Alternatively, a service can be used by starting any browser on the caregiver terminal 20 and the care recipient terminal 30 and logging into a user-dedicated website.

A caregiver CG can propose an addable service to a care recipient CP and/or a relative F thereof by causing the caregiver terminal 20 to display the service. On the other hand, the relative F of the care recipient CP can request addition of a service to the caregiver CG by causing the care recipient terminal 30 to display an addable service.

The providing unit 108 may determine and provide an addable service by further using a service required for a care recipient CP. For example, the providing unit 108 may determine a service, based on a care level of the care recipient CP.

Furthermore, the providing unit 108 may also provide a cost required for the addable service. For example, a cost required for a service out of a list of a plurality of addable services provided by the providing unit 108, selection of the service being accepted, may be computed and provided.

Further, the providing unit 108 may check a schedule of a caregiver CG in charge of the care recipient CP and execute the determination processing of an addable service, based on availability in the schedule. When there is room in the schedule of the caregiver CG, the determination processing of an addable service is executed. When there is no room in the schedule of the caregiver CG, the determination processing is not executed.

Furthermore, the providing unit 108 may determine an addable service, based on a nursing care insurance points of a care recipient CP. Specifically, when the total points at the present point in time is less than an upper limit of the nursing care insurance points of the care recipient CP in a predetermined period, the determination processing of an addable service is executed. When the total points at the present point in time is not less than the upper limit of the nursing care insurance points of the care recipient CP in the predetermined period, the determination processing is not executed.

According to the present example embodiment, the providing unit 108 can provide an addable service on the appointed day by using a qualification of a caregiver CG and a care plan, and therefore an additional service can be suitably provided for a care recipient CP when, for example, there is an excess of time at the spot.

### Fourth Example Embodiment

### <Functional Configuration Example>

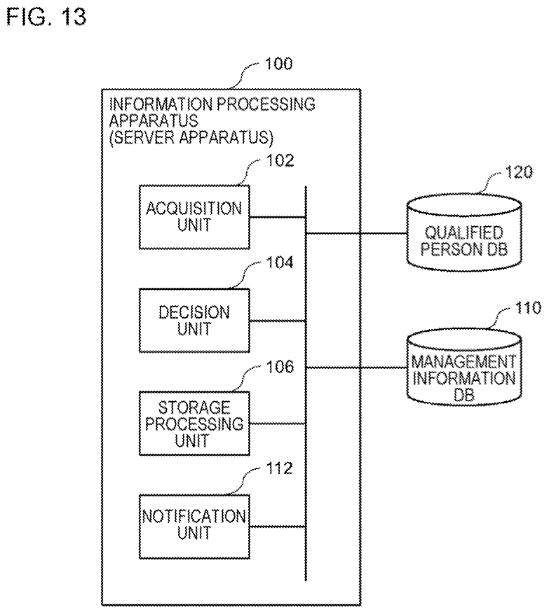

FIG. 13 is a functional block diagram logically illustrating a configuration of a server apparatus (information processing apparatus) 100 according to the present example embodiment. The server apparatus 100 according to the present example embodiment is similar to one of the aforementioned example embodiments except for being configured to make notification to an apparatus of a provider when a criterion is not satisfied in a decision unit 104. The information processing apparatus 100 in FIG. 13 includes an acquisition unit 102, the decision unit 104, and a storage processing unit 106 that are the same as those in the information processing apparatus 100 in FIG. 1 and further includes a notification unit 112. The example in FIG. 13 illustrates an example combined with the configuration of the information processing apparatus 100 according to the first example embodiment in FIG. 1. Note that the server apparatus 100 according to the present example embodiment may be combined with at least one of the configurations according to other example embodiments without contradicting each other.

When a criterion is not satisfied in the decision unit 104, the notification unit 112 makes notification to an apparatus of a provider (the care provider terminal 10 in FIG. 6).

The notification method includes at least one of displaying a screen on a display of the care provider terminal 10, transmitting a mail or a message, and outputting a voice or a warning sound from a speaker. At least one destination out of a mail address being a destination of a mail or a message, an IP address of a mobile terminal, and a mobile phone number may be preregistered.

Examples of a notification timing are described below but are not limited thereto.

(1) When a caregiver CG visits a house of a care recipient CP and a decision result is acquired
(2) Periodically (at a fixed time, every day, every week, or every month)

In the case of aforementioned (1), when deciding that a criterion is not satisfied, the decision unit 104 hands over the decision result to the notification unit 112. Then, when receiving the decision result from the decision unit 104, the notification unit 112 determines a corresponding care provider and makes notification to the care provider terminal 10 of the care provider.

In the case of aforementioned (2), after the storage processing unit 106 stores a decision result by the decision unit 104 into the management information database 110, the notification unit 112 periodically checks the management information database 110. The notification unit 112 retrieves a record indicating that a decision result does not satisfy a criterion from the management information database 110. For example, the notification unit 112 extracts a record with an eligible person flag being 0. Then, the notification unit 112 determines a caregiver ID of the record. Furthermore, the notification unit 112 determines a care provider to which the caregiver ID belongs. Then, the notification unit 112 makes notification to the care provider terminal 10 of the determined care provider.

According to the present example embodiment, the notification unit 112 can make notification to the care provider terminal 10 of a care provider when a decision result by the decision unit 104 does not satisfy a criterion, and therefore the care provider can recognize a wrongful act such as impersonation of a caregiver CG. The care provider receiving the notification can take suitable measures.

### Fifth Example Embodiment

### <Functional Configuration Example>

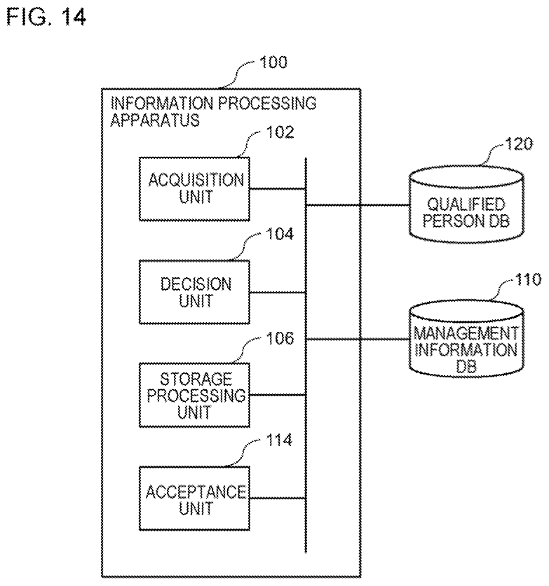

FIG. 14 is a functional block diagram logically illustrating a configuration of a server apparatus (information processing apparatus) 100 according to the present example embodiment. The server apparatus 100 according to the present example embodiment is similar to one of the aforementioned example embodiments except for being configured to record a care content performed on the appointed day. The information processing apparatus 100 in FIG. 14 includes an acquisition unit 102, a decision unit 104, and a storage processing unit 106 that are the same as those in the information processing apparatus 100 in FIG. 1 and further includes an acceptance unit 114. The example in FIG. 14 illustrates an example combined with the configuration of the information processing apparatus 100 according to the first example embodiment in FIG. 1. Note that the server apparatus 100 according to the present example embodiment may be combined with at least one of the configurations according to other example embodiments without contradicting each other.

The acceptance unit 114 accepts information indicating a work content performed on the appointed day.

The storage processing unit 106 causes a management information database 110 to store the accepted information indicating the work content as care history information 150.

The decision unit 104 decides whether the work content is corresponded to the qualification of the caregiver CG.

The storage processing unit 106 causes the management information database 110 to store the decision result.

Information indicating a work content at least includes a care item.

The decision unit 104 acquires a qualification corresponding to the accepted care item with reference to the care information by qualification 130 in FIG. 7 and decides whether a level of the qualification of the caregiver CG satisfies a criterion for the acquired qualification.

For example, the acceptance unit 114 may accept a response to a questionnaire on a care content or a caregiver CG from a care recipient CP or a relative F of the care recipient CP. For example, a response can be accepted by causing a care recipient terminal 30 to display a response screen to a questionnaire. Acceptance of a response to a questionnaire may be performed in either one of a selective style and a free description style. Text data entered in a free description field may be recorded as-is into the care history information 150 to be described later.

The decision unit 104 may compile questionnaire responses and perform scoring for each caregiver CG. For example, the decision unit 104 may cause selection of a satisfaction level for each care item in a plurality of grades (such as satisfactory, rather satisfactory, fair, rather unsatisfactory, and unsatisfactory), compile the responses with a score assigned to each grade, and compute a customer satisfaction level for each caregiver CG.

It is preferable that at least a care manager CM can browse the scoring result on a care provider terminal 10. Furthermore, a caregiver CG himself/herself may be able to browse the result on a caregiver terminal 20. Furthermore, the result may be browsed on the care recipient terminal 30 of a care recipient CP or a relative F of the care recipient CP as an evaluation of each caregiver CG, and a caregiver CG requested for the next and later visits may be specified.

The acquisition unit 102 further acquires information about care of a care recipient CP. For example, the acquired information includes a date and time of the visit, the name of the care recipient CP, a care item, the name of a visiting caregiver CG, a starting time and an ending time of work, a report by the caregiver CG, and a voice from the care recipient CP.

The storage processing unit 106 causes the management information database 110 to store a history of care for each care recipient CP as the care history information 150.

The acceptance unit 114 can accept the report by the caregiver CG and the voice from the care recipient CP.

A report by a caregiver CG can be input from an input screen on the caregiver terminal 20. The caregiver terminal 20 accepts a report input by a care recipient CP and transmits the report to the server apparatus 100. Alternatively, the caregiver terminal 20 may upload the report to a user-dedicated web page.

The voice from the care recipient CP can be input from an input screen on the care recipient terminal 30 by the care recipient CP or a relative F of the care recipient CP. The voice from the care recipient CP may be accepted as a response in the aforementioned questionnaire style or may be accepted as a free description. The care recipient terminal 30 accepts the voice from the care recipient CP input by the care recipient CP or the relative F of the care recipient CP and transmits the voice to the server apparatus 100. Alternatively, the care recipient terminal 30 may upload the report to a user-dedicated web page.

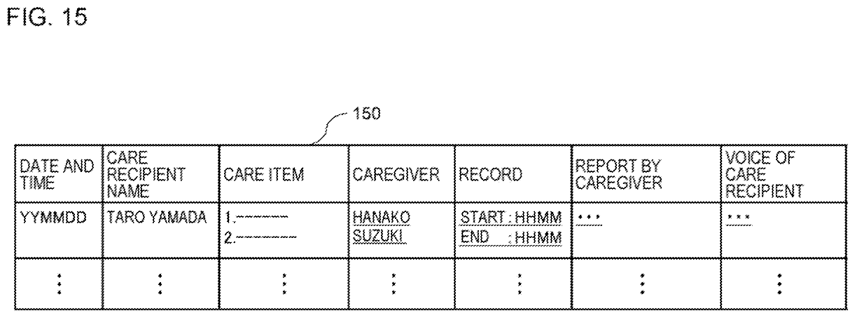

FIG. 15 is a diagram illustrating an example of a data structure of the care history information 150.

The care history information 150 includes a date, the name of a care recipient CP, a care item, the name of a visiting caregiver CG, a starting time and an ending time of work, a report by the caregiver CG, and a voice from the care recipient CP but is not limited thereto.

The care history information 150 may be displayed on the care provider terminal 10, the caregiver terminal 20, and the care recipient terminal 30. Specifically, a care manager CM, a caregiver CG, a care recipient CP, and a relative F of the care recipient CP can browse the care history information 150.

According to the present example embodiment, the storage processing unit 106 accumulates a history of care information, and therefore a care manager CM, a caregiver CG, a care recipient CP, and a relative F of the care recipient CP can browse the care history information 150 by using the care provider terminal 10, the caregiver terminal 20, and the care recipient terminal 30. Thus, safety and security of the care recipient CP can be guaranteed. Furthermore, the relative F of the care recipient CP can also check security of home-visit caregiving.

### Sixth Example Embodiment

### <Functional Configuration Example>

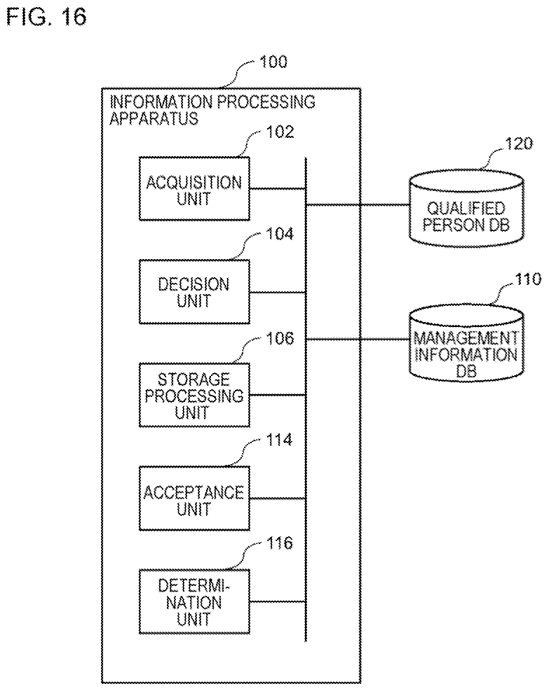

FIG. 16 is a functional block diagram logically illustrating a configuration of a server apparatus (information processing apparatus) 100 according to the present example embodiment. The server apparatus 100 according to the present example embodiment is similar to the aforementioned sixth example embodiment except for being configured to determine care status by using an image from a camera. The information processing apparatus 100 in FIG. 16 includes an acquisition unit 102, a decision unit 104, a storage processing unit 106, and an acceptance unit 114 that are the same as those in the server apparatus 100 in FIG. 14 and further includes a determination unit 116. The example in FIG. 16 illustrates an example combined with the configuration of the information processing apparatus 100 according to the first example embodiment in FIG. 1. Note that the server apparatus 100 according to the present example embodiment may be combined with at least one of the configurations according to other example embodiments without contradicting each other.

The determination unit 116 determines care status by using an image from a camera.

The acceptance unit 114 accepts information indicating care status determined by the determination unit 116 as information indicating a work content performed on the appointed day. The accepted information is stored into a management information database 110 as care history information 150 by the storage processing unit 106.

Examples of information indicating care status include information indicating a work content of actually performed care, information indicating whether work corresponding to a care item included in the care plan is performed, and information indicating whether an action or an act irrelevant to care, such as a wrongful act or a criminal act is performed. Furthermore, information indicating care status may include an image in which care status is captured.

An image in which care status is captured may be acquired by using a body-worn camera. For example, a caregiver CG may wear a body-worn camera and capture an image of a state of a care recipient CP during care. Alternatively, a care recipient CP may wear a body-worn camera and capture an image of a state of care by a caregiver CG providing the care. Further, the aforementioned acquisition unit 102 may acquire biometric information of the caregiver CG by using the body-worn camera.

Examples of a method for determining care status include the following but are not limited thereto. A plurality of the following items may be combined.

(1) Preregistering feature information of a characteristic action or a characteristic item image (for example, items such as a toothbrush, a tooth powder, a cup, and a saucer in a case of toothbrushing) in work corresponding to each care item in the management information database 110 as care work content information (unillustrated) and determining work from the captured image by image analysis. Specifically, a work content of care is determined as information indicating care status.
(2) Retrieving and determining whether feature information of work for a care item included in a care plan is included in a captured image by image analysis. Specifically, information indicating whether the work for the care item included in the care plan is performed is determined as information indicating care status.
(3) Preregistering feature information of an action being another surveillance target irrelevant to care work in the management information database 110 as surveillance target action information and detecting the another action from a captured image by image analysis. Examples of an action being a surveillance target include a conversation content, a recess, violence, violent language, neglect, a theft, and a criminal act. Information indicating that an action irrelevant to care, such as a wrongful act or a criminal act is being performed or that such an act is not being performed may be determined as care status.

Further, a caregiver CG or a care recipient CP may be caused to utter a care content being performed (such as “I am brushing my teeth now.” or “I will go for a walk from now.”), and the determination unit 116 may collect the voice and determine information indicating care status by voice analysis processing.

The storage processing unit 106 may store an image in which care status is captured, the image being accepted by the acceptance unit 114, into the management information database 110. Alternatively, the image may be transmitted to a care provider terminal 10 or a care recipient terminal 30 by an authentication terminal 5 or another communication means (unillustrated). Video image delivery may be performed in real time during care time. Note that video image delivery may be delayed. A care recipient CP or a relative F of the care recipient CP may be able to browse the image on a user-dedicated web page by using the care recipient terminal 30.

Then, the decision unit 104 decides whether the determined care status matches the care plan.

For example, when the care status is decided not to match the care plan by the decision unit 104, the notification unit 112 may make notification to a care provider. The notification method may be similar to that according to the aforementioned fourth example embodiment.

According to the present example embodiment, the determination unit 116 determines care status by using an image from a camera, and therefore an image can be left as an evidence serving as a deterrent against a wrongful act. A care recipient CP and a relative F of the care recipient CP can feel more relieved when receiving home-visit caregiving.

While the example embodiments of the present invention have been described above with reference to the drawings, the example embodiments are exemplifications of the present invention, and various configurations other than those described above may be employed.

For example, fingerprint authentication or face authentication processing may be performed by using a caregiver terminal 20 of a caregiver CG instead of using the authentication terminal 5. Then, the caregiver terminal 20 may include a Global Positioning System (GPS) receiver, receive a current position from the GPS, and transmit the current position to the server apparatus 100 along with biometric information. Note that a positioning system is not limited thereto. Another positioning system [such as the Quasi-Zenith Satellite System (QZSS)] may also be used.

The decision unit 104 may decide whether the acquired positional information matches the address of a care recipient CP planned to be visited. The storage processing unit 106 may cause the management information database 110 to store the decision result along with the acquired positional information.

A camera may be installed on a bed of a care recipient CP. A facial image or the like may be acquired by using an image from the camera as biometric information of a visiting caregiver CG and be transmitted to the server apparatus 100. Image capture may be performed periodically.

While the present invention has been described with reference to example embodiments and examples thereof, the present invention is not limited to the aforementioned example embodiments and examples. Various changes and modifications that may be understood by a person skilled in the art may be made to the configurations and details of the present invention without departing from the scope of the present invention.

Note that, when information about a user is acquired and used in the present invention, the acquisition and the use are assumed to be performed legally.

The whole or part of the example embodiments disclosed above may also be described as, but not limited to, the following supplementary notes.

1. An information processing apparatus including:

an acquisition unit that acquires biometric information of a visiting caregiver;

a decision unit that reads a qualification of the caregiver corresponding to the acquired biometric information from a storage unit that stores the biometric information and a qualification in association with each other and decides whether the qualification satisfies a criterion; and a storage processing unit that causes a storage unit to store the decision result.

2. The information processing apparatus according to 1., wherein

care information indicating a level of the qualification is set based on a care plan of an appointed day, and

the decision unit decides whether the qualification of the caregiver satisfies a level of the qualification.

3. The information processing apparatus according to 2., further including

a providing unit that determines and provides an addable service corresponding to the qualification of the caregiver by using a level of a qualification, the level being set for each care content, and a care plan.

4. The information processing apparatus according to 3., wherein

the providing unit determines and provides an addable service by further using a service required for a care recipient.

5. The information processing apparatus according to any one of 1. to 4., wherein

the acquisition unit further acquires information about care of a care recipient, and

the storage processing unit causes the storage unit to store a history of care for each care recipient.

6. The information processing apparatus according to any one of 1. to 5., further including

a notification unit that makes notification to an apparatus of a provider when the criterion is not satisfied in the decision unit.

7. The information processing apparatus according to any one of 1. to 6., further including

an acceptance unit that accepts information indicating a work content performed on an appointed day, wherein

the storage processing unit causes the storage unit to store accepted information indicating the work content,

the decision unit decides whether the work content is corresponded to the qualification of the caregiver, and

the storage processing unit causes the storage unit to store the decision result.

8. The information processing apparatus according to 7., further including

a determination unit that determines care status by using an image from a camera, wherein

the acceptance unit accepts the care status determined by the determination unit.

9. The information processing apparatus according to 8., wherein

the decision unit decides whether the determined care status matches a care plan.

10. A home-visit caregiving service system including:

a biometric information acquisition apparatus that acquires biometric information of a caregiver at a place being visited; and

a server apparatus, wherein

the server apparatus includes:
an acquisition unit that acquires biometric information of the visiting caregiver by using the biometric information acquisition apparatus;
a decision unit that reads a qualification of the caregiver corresponding to the acquired biometric information from a storage unit that stores the biometric information and a qualification in association with each other and decides whether the qualification satisfies a criterion; and
a storage processing unit that causes a storage unit to store the decision result.
   11. The home-visit caregiving service system according to 10., wherein

care information indicating a level of the qualification is set based on a care plan of an appointed day, and

the decision unit in the server apparatus decides whether the qualification of the caregiver satisfies a level of the qualification.

12. The home-visit caregiving service system according to 11., wherein

the server apparatus further includes
a providing unit that determines and provides an addable service corresponding to the qualification of the caregiver by using a level of a qualification, the level being set for each care content, and a care plan.
   13. The home-visit caregiving service system according to 12., wherein

the providing unit in the server apparatus determines and provides an addable service by further using a service required for a care recipient.

14. The home-visit caregiving service system according to any one of 10. to 13., wherein

the acquisition unit in the server apparatus further acquires information about care of a care recipient, and

the storage processing unit in the server apparatus causes the storage unit to store a history of care for each care recipient.

15. The home-visit caregiving service system according to any one of 10. to 14., wherein

the server apparatus further includes
a notification unit that makes notification to an apparatus of a provider when the criterion is not satisfied in the decision unit.
   16. The home-visit caregiving service system according to any one of 10. to 15., wherein

the server apparatus further includes an acceptance unit that accepts information indicating a work content performed on an appointed day,

the storage processing unit in the server apparatus causes the storage unit to store accepted information indicating the work content,

the decision unit in the server apparatus decides whether the work content is corresponded to the qualification of the caregiver, and

the storage processing unit in the server apparatus causes the storage unit to store the decision result.

17. The home-visit caregiving service system according to 16., wherein

the server apparatus further includes a determination unit that determines care status by using an image from a camera, and

the acceptance unit in the server apparatus accepts the care status determined by the determination unit.

18. The home-visit caregiving service system according to 17., wherein

the decision unit in the server apparatus decides whether the determined care status matches a care plan.

19. An information processing method including, by an information processing apparatus:

acquiring biometric information of a visiting caregiver;

reading a qualification of the caregiver corresponding to the acquired biometric information from a storage unit that stores the biometric information and a qualification in association with each other and deciding whether the qualification satisfies a criterion; and

causing a storage unit to store the decision result.

20. The information processing method according to 19., wherein

care information indicating a level of the qualification is set based on a care plan of an appointed day, and

the information processing method further includes, by the information processing apparatus,
deciding whether the qualification of the caregiver satisfies a level of the qualification.
   21. The information processing method according to 20., further including, by the information processing apparatus,

determining and providing an addable service corresponding to the qualification of the caregiver by using a level of a qualification, the level being set for each care content, and a care plan.

22. The information processing method according to 21., further including, by the information processing apparatus,

determining and providing an addable service by further using a service required for a care recipient.

23. The information processing method according to any one of 19. to 22., further including, by the information processing apparatus:

further acquiring information about care of a care recipient; and

causing the storage unit to store a history of care for each care recipient.

24. The information processing method according to any one of 19. to 23., further including, by the information processing apparatus,

making notification to an apparatus of a provider when the criterion is not satisfied.

25. The information processing method according to any one of 19. to 24., further including, by the information processing apparatus:

accepting information indicating a work content performed on an appointed day;

causing the storage unit to store accepted information indicating the work content;

deciding whether the work content is corresponded to the qualification of the caregiver; and

causing the storage unit to store the decision result.

26. The information processing method according to 25., further including, by the information processing apparatus:

determining care status by using an image from a camera; and

accepting the determined care status.

27. The information processing method according to 26., further including, by the information processing apparatus,

deciding whether the determined care status matches a care plan.

28. A program for causing a computer to execute:

a procedure for acquiring biometric information of a visiting caregiver;

a procedure for reading a qualification of the caregiver corresponding to the acquired biometric information from a storage unit that stores the biometric information and a qualification in association with each other and deciding whether the qualification satisfies a criterion; and

a procedure for causing a storage unit to store the decision result.

29. The program according to 28., wherein

care information indicating a level of the qualification is set based on a care plan of an appointed day, and

the program further causes the computer to execute
a procedure for deciding whether a qualification of the caregiver satisfies a level of the qualification.
   30. The program according to 29., further causing the computer to execute

a procedure for determining and providing an addable service corresponding to a qualification of the caregiver by using a level of a qualification, the level being set for each care content, and a care plan.

31. The program according to 30., further causing the computer to execute

a procedure for determining and providing an addable service by further using a service required for a care recipient.

32. The program according to any one of 28. to 31., further causing the computer to execute:

a procedure for further acquiring information about care of a care recipient; and

a procedure for causing the storage unit to store a history of care for each care recipient.

33. The program according to any one of 28. to 32., further causing the computer to execute

a procedure for making notification to an apparatus of a provider when the criterion is not satisfied.

34. The program according to any one of 28. to 33., further causing the computer to execute:

a procedure for accepting information indicating a work content performed on an appointed day;

a procedure for causing the storage unit to store accepted information indicating the work content;

a procedure for deciding whether the work content is corresponded to the qualification of the caregiver; and

a procedure for causing the storage unit to store the decision result.

35. The program according to 34., further causing the computer to execute:

a procedure for determining care status by using an image from a camera; and

a procedure for accepting the determined care status.

36. The program according to 35., further causing the computer to execute

a procedure for deciding whether the determined care status matches a care plan.

### REFERENCE SIGNS LIST

1 Home-visit caregiving service system
3 Communication network
5 Authentication terminal
10 Care provider terminal
20 Caregiver terminal
30 Care recipient terminal
50 Qualified person database
100 Information processing apparatus (server apparatus)
200 Coordination apparatus
102 Acquisition unit
104 Decision unit
106 Storage processing unit
108 Providing unit
112 Notification unit
114 Acceptance unit
116 Determination unit
110 Management information database
120 Qualified person database
130 Care information by qualification
132 Qualification information by item
140 Care plan information
150 Care history information
1000 Computer
1010 Bus
1020 Processor
1030 Memory
1040 Storage device
1050 Input-output interface
1060 Network interface

What is claimed is:

1. An information processing apparatus comprising:
at least one memory configured to store instructions; and
at least one processor configured to execute the instructions to:
acquire biometric information of a visiting caregiver;
read a qualification of the caregiver corresponding to the acquired biometric information from a storage unit that stores the biometric information and a qualification in association with each other and decide whether the qualification satisfies a criterion; and
cause a storage unit to store the decision result.

2. The information processing apparatus according to claim 1, wherein
care information indicating a level of the qualification is set based on a care plan of an appointed day, and
wherein the at least one processor is further configured to execute the instructions to
decide whether the qualification of the caregiver satisfies a level of the qualification.

3. The information processing apparatus according to claim 2,
wherein the at least one processor is further configured to execute the instructions to
determine and provide an addable service corresponding to the qualification of the caregiver by using a level of a qualification, the level being set for each care content, and a care plan.

4. The information processing apparatus according to claim 3, wherein
the at least one processor is further configured to execute the instructions to
determine and provide an addable service by further using a service required for a care recipient.

5. The information processing apparatus according to claim 1, wherein
the at least one processor is further configured to execute the instructions to:
further acquire information about care of a care recipient, and
cause the storage unit to store a history of care for each care recipient.

6. The information processing apparatus according to claim 1,
wherein the at least one processor is further configured to execute the instructions to
make notification to an apparatus of a provider when the criterion is not satisfied.

7. The information processing apparatus according to claim 1,
wherein the at least one processor is further configured to execute the instructions to:
accept information indicating a work content performed on an appointed day, wherein
cause the storage unit to store accepted information indicating the work content,
decide whether the work content is corresponded to the qualification of the caregiver, and
cause the storage unit to store the decision result.

8. The information processing apparatus according to claim 7,
wherein the at least one processor is further configured to execute the instructions to:
determine care status by using an image from a camera, wherein
accept the care status determined.

9. The information processing apparatus according to claim 8, wherein
the at least one processor is further configured to execute the instructions to
decide whether the determined care status matches a care plan.

10.-18. (canceled)

19. An information processing method comprising, by an information processing apparatus:
acquiring biometric information of a visiting caregiver;
reading a qualification of the caregiver corresponding to the acquired biometric information from a storage unit that stores the biometric information and a qualification in association with each other and deciding whether the qualification satisfies a criterion; and
causing a storage unit to store the decision result.

20.-27. (canceled)

28. A non-transitory computer-readable storage medium storing a program for causing a computer to execute:
a procedure for acquiring biometric information of a visiting caregiver;
a procedure for reading a qualification of the caregiver corresponding to the acquired biometric information from a storage unit that stores the biometric information and a qualification in association with each other and deciding whether the qualification satisfies a criterion; and
a procedure for causing a storage unit to store the decision result.

29.-36. (canceled)
